(19) United States
(12) Patent Application Publication
(10) Pub. No.: US 2011/0196360 A1
(43) Pub. Date: Aug. 11, 2011

(54) Hand manipulative dermatological instrument for blowing air across wounds

(76) Inventor: Michael McCatty, Orland Park, IL (US)

(21) Appl. No.: 12/980,886

(22) Filed: Dec. 29, 2010

Related U.S. Application Data
(60) Provisional application No. 61/291,473, filed on Dec. 31, 2009.

(51) Int. Cl. A61B 18/04

(52) U.S. Cl. 606/27

(57) ABSTRACT

A handheld cauter comprising a casing made from a durable material. The handheld cauter comprising a blowing device mounted inside the casing and a heating device also mounted firmly within the casing. The blowing device and the heating device being positioned such that the blowing device is capable of blowing air across the heating device. By having constricting internal walls, the casing of the cauter directs air out of the discharge end in conjunction with the air motion caused by the blowing device. The cauter is capable of being manipulated in such a way as to direct air onto a wound.

### CROSS-REFERENCE TO RELATED APPLICATIONS

This application claims the benefit of U.S. Provisional Application No. 61/291,473, filed Dec. 31, 2009.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND OF THE INVENTION

(1) Field of the Invention

This invention relates generally to cauters; by “cauter” it is meant to include the commonly accepted definition as well as a device to apply a flow of air, the air being heated or not, to a wound as an alternative to direct contact with a heated surface such as metal. More particularly, the invention relates to hand manipulative air blowing devices, including without limitation electric heating devices, and dermatological surgical instruments that blow air.

It is clear that the device would be useful for a skin wound repair operation wherein air, heated or not, is directed onto a wound thereby decreasing the blood flow from the wound. In a similar manner, a device would be useful for sanitizing wounds; by “wound” it is meant to include the commonly accepted definition as well as sensitive skin areas such as the inner lining of the nasal passage, mouth, or ear to name a few examples.

(2) Description of Related Art Including Information Disclosed Under 37 CFR 1.97 and 37 CFR 1.98.

It is well known that surgeons use cauters to apply heat to wounds and cause coagulation to stop bleeding. Hippocratus made reference to the cauter as early as 400 B.C.

Human skin will generally cauterize from heat at 150 degrees fahrenheit. Traditionally, the cauter makes physical contact with the skin to do its work. It is desirable to use air, including without limitation heated air, to cauterize wounds.

The prior art discloses many devices that blow air, including heated air, across a body. There are, for instance, salve applicators which blow air across a heated surface and direct the air toward a body as shown by U.S. Pat. No. 4,596,965 (issued Jun. 24, 1986). Handheld portable body dryers are used to totally dry skin as shown by U.S. Pat. No. 5,394,620 (issued Mar. 7, 1995). Shavers are made to blow heated air across skin to give a better cut as shown by U.S. Pat. No. 5,394,777 (issued Mar. 7, 1995).

The prior art fails to disclose an air blowing device that is handheld and capable of cauterization. Accordingly, a device such as herein disclosed would be useful because it fills a need in the industry for a device that uses air, hot or otherwise, to cauterize. The increased flow and temperature of the air coming into contact with a wound will cause cauterization. If the air is not heated, then the rapid air flow of air itself over a wound will cause the wound to become sanitized by diminishing moisture. It is clear that a device that serves a need for such a cauter to be handheld and capable of use by any person, not just for use by surgeons, would be useful.

An inventive cauter is provided comprising a casing that is suited for being easily carried or manipulated. The portable casing comprising within a blowing device, such as a fan, and a heating device. The casing is formed such that one of its ends is narrower than the casing generally.

### BRIEF SUMMARY OF THE INVENTION

In some embodiments my invention is directed to a handheld cauter comprising a casing, the casing having a discharge end, a fan, a heating device, the fan being mounted within the casing, the heating device being mounted within the casing, the heating device and the fan being positioned such that the fan is capable of blowing air toward the heating device, the discharge end having an opening, and a means for directing the air through the opening and onto a wound.

In some embodiments my invention is directed to a handheld cauter comprising, a casing, the casing having a discharge end, a fan, a heating device, the heating device being switchably powered, the fan being removably mounted within the casing, the heating device being removably mounted within the casing, the heating device and the fan being positioned such that the fan is capable of blowing air toward the heating device, the discharge end having an opening, the heating device being capable of producing heat energy from an energy source chosen from the group including electrical, gas, solar, nuclear, and chemical, and a means for directing the air through the opening and onto a wound.

In some embodiments my invention is directed to a handheld cauter comprising a casing, the casing having a discharge end, a fan, a heating device, the heating device being switchably powered, the fan being removably mounted within the casing, the heating device being removably mounted within the casing, the heating device and the fan being positioned such that the fan is capable of blowing air toward the heating device, the discharge end having an opening, the heating device being capable of producing heat energy from an energy source chosen from the group including electrical, gas, solar, nuclear, and chemical, a means for directing the air through the opening and onto a wound, the air being capable of being increased in temperature thereby accelerating the healing of the wound, the air being capable of increased flow thereby diminishing moisture and sanitizing the wound, and the casing having a length between 4 and 9 inches.

### BRIEF DESCRIPTION OF THE SEVERAL VIEWS OF THE DRAWING

FIG. 1 shows diagrammatically a hand manipulative device according to the prior art.

FIG. 2 shows diagrammatically a handheld cauter according to the present invention.

FIG. 3 shows diagrammatically a handheld cauter according to the present invention being used by a person with a wound.

### DETAILED DESCRIPTION OF THE INVENTION

Some embodiments of my invention are capable of comprising a casing containing essential components. The casing is made from a durable material, such as plastic, to name one example. The casing is small enough to be held in one hand. The casing looks similar in shape to handheld ear and nose hair trimmers often seen publicly advertised, though it could be made to look different than these, more pen-like for instance, without taking away from its essential features. Preferably, the casing is sized to be easy to hold. A casing that is between 4 and 9 inches long is preferred. More preferably, the casing may be between 4¾ inches and 8¼ inches. Most preferably, the casing may be no longer than 8 inches which facilitates hand holding of my invention, and cooperation and operation of the components, e.g. a shorter length can cause the air exiting from the casing to be uncomfortably hot.

In some embodiments of my invention, one component contained inside the casing is a fan. The fan is mounted inside the casing such that it remains firmly in place during operation. However, it is not contrary to the object of my invention that the fan is capable of being removed from within the casing, to be cleaned or replaced, for example. The means for operating the fan, though not essential to my invention, can be by electricity or manually to name a few examples.

Inside the casing of some embodiments of my invention is mounted a heating device. Similarly to the fan, the heating device remains firmly in place during operation but is capable of being removed from within the casing. The heating device can be operated independently of the fan, i.e, a user can turn the fan on and have the heating device turned off. However, it is not contrary to the object of my invention that the fan and heating device are operated together, i.e, both on or both off.

The heating device of my invention produces heat energy from an energy source chosen from the group including electrical, gas, solar, nuclear, or chemical.

For some embodiments of my invention the fan and the heating device are mounted in a position such that the fan is capable of blowing air across the heating device. If the heating device is in operation, then the air will rise in temperature.

The casing of some embodiments of my invention are comprising a discharge end where air exits from within through a hole that is designed into the discharge end. By having constricting internal walls, though other means may be used, the casing of my invention directs the air through the hole in the discharge end in conjunction with the air motion caused by a blowing device.

A user of some embodiments of my invention can hold it in one hand and manipulate the invention in such a way as to direct increased volumes of air onto a wound. I have discovered that doing so causes the blood flow from the wound to decrease or completely stop or causes the wound to become sanitized, I believe that sanitizing of the ear and or nasal canals can help cure and or prevent a cold or flu.

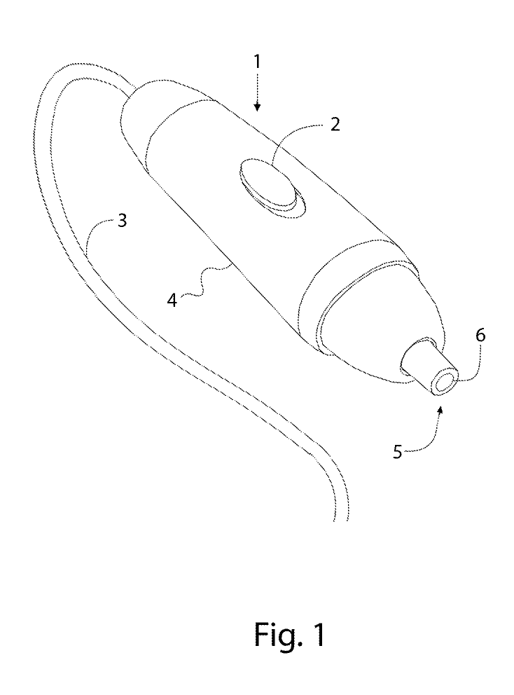

Referring now to the drawing figures, particularly to FIG. 1, there is illustrated a hand manipulative device, generally designated 1. The various elements of the hand manipulative device are well known in the art and a detailed description of the device is not necessary. Suffice it to say that the various elements of the hand manipulative device 1 comprising a casing 4 manufactured of a durable material. Electrically connected to the hand manipulative device 1 is an electrical cord 3. The case 4 of the hand manipulative device includes a discharge end 5 with an opening 6, and a switch 2 that controls the supply of electrical energy, via the electrical cord 3, to electrical components operating within the casing 4.

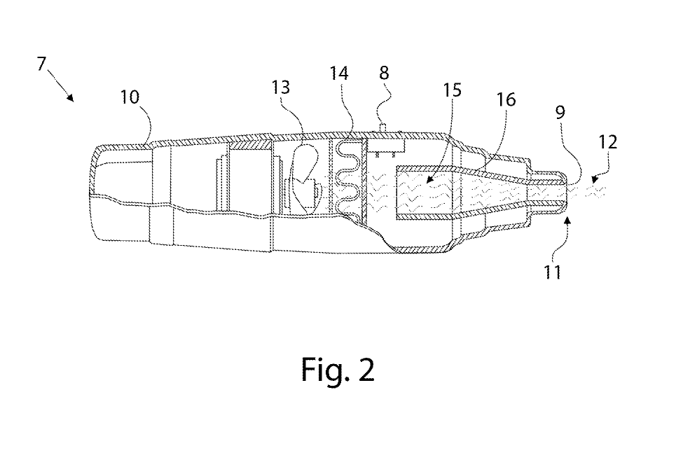

In FIG. 2 we have a diagram of a handheld cauter 7. Depicted is at least one switch 8, which is well known in the art. The casing 10 of the handheld cauter 7 is narrow at a discharge end 11. At the discharge end 11 there is a hole 9 that allows escaping air 12 to exit from within the casing 10.

Inside the casing 10 of the handheld cauter 7 are components including a blowing device, such as a fan 13, and a heating device, such as an electric heating element 14. The means for supplying power, which can be from any source such as battery or a/c for example, and the electrical componentry for the blowing device 13 is well known in the art.

The blowing device 13 causes air within 15 the casing 10 to move toward the discharge end 11 and exit the casing 10 through the hole 9. The blowing device 13 directs the air through the heating device 14 before the air within 15 the casing 10 exits through the hole 9.

At least one switch 8 is capable of turning off or turning on the blowing device 13 and the heating device 14, together or independently of each other. When the heating device 14 is turned on, the air within 15 the casing 10 will rise in temperature and any air that passes through or around the heating device 14, as it is blown by the blowing device 13, will rise further in temperature. The air within 15 the casing 10 is directed out through the hole 9 in the discharge end 11 via constricted internal walls 16. The escaping air 12 that exits from the casing 10 at the hole 9 is therefore either warmed air or not.

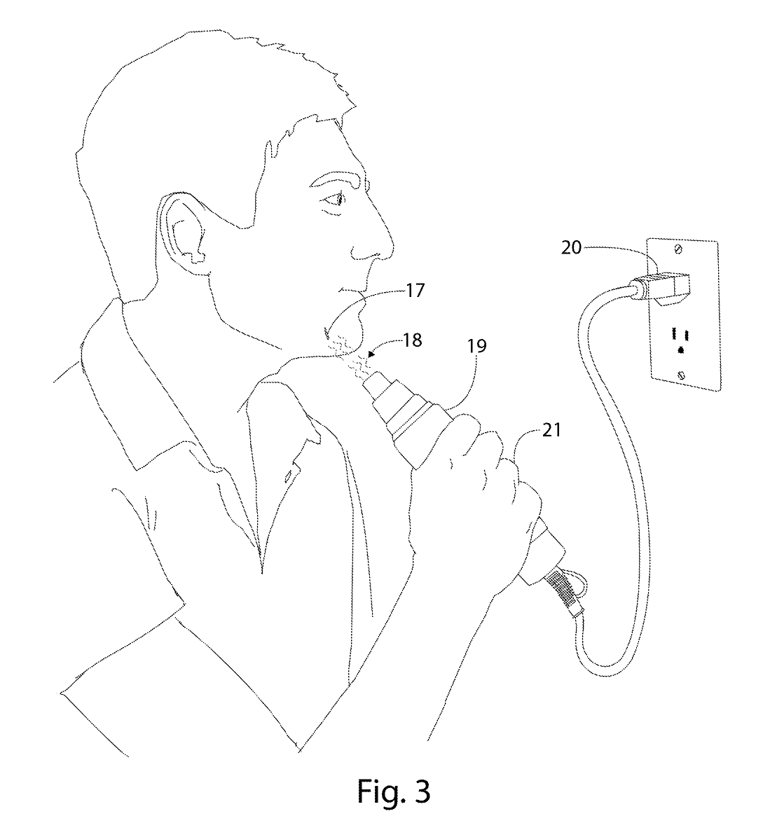

In FIG. 3 is depicted a handheld cauter 19 being used to direct air towards a wound 17. The handheld cauter 19 is powered by an alternating current source via the electrical cord 20. The user holds the device in the user's hand 21, thereby easily manipulating the handheld cauter 19. The air that exits 18 from the handheld cauter 19 is directed toward the wound 17 and accelerates the healing of the wound 17.

I claim:

1. A handheld cauter comprising:
(a) a casing;
(b) the casing having a discharge end;
(c) a fan;
(d) a heating device;
(e) the fan being mounted within the casing;
(f) the heating device being mounted within the casing;
(g) the heating device and the fan being positioned such that the fan is capable of blowing air toward the heating device;
(h) the discharge end having an opening;
(i) a means for directing the air through the opening and onto a wound.

2. The cauter of claim 1 wherein the casing has a length between 4 and 9 inches.

3. The cauter of claim 1 wherein the casing has a length between 4.75 and 8.25 inches.

4. The cauter of claim 1 wherein the heating device is switchably powered.

5. The cauter of claim 1 wherein the heating device produces heat energy from an energy source chosen from the group including electrical, gas, solar, nuclear, and chemical.

6. The cauter of claim 1 wherein the air is capable of increased flow thereby diminishing moisture and sanitizing the wound.

7. The cauter of claim 1 wherein the air is capable of being increased in temperature thereby accelerating the healing of the wound.

8. A handheld cauter comprising:
(a) a casing;
(b) the casing having a discharge end;
(c) a fan;
(d) a heating device;
(e) the heating device being switchably powered;
(f) the fan being removably mounted within the casing;
(g) the heating device being removably mounted within the casing;
(h) the heating device and the fan being positioned such that the fan is capable of blowing air toward the heating device;
(i) the discharge end having an opening;
(j) the heating device being capable of producing heat energy from an energy source chosen from the group including electrical, gas, solar, nuclear, and chemical;
(k) a means for directing the air through the opening and onto a wound.

9. The cauter of claim 8 wherein the air is capable of being increased in temperature thereby accelerating the healing of the wound.

10. The cauter of claim 8 wherein the air is capable of increased flow thereby diminishing moisture and sanitizing the wound.

11. The cauter of claim 8 wherein the casing has a length between 4 and 9 inches.

12. The cauter of claim 8 wherein the casing has a length between 4.75 and 8.25 inches.

13. A handheld cauter comprising:
(a) a casing;
(b) the casing having a discharge end;
(c) a fan;
(d) a heating device;
(e) the heating device being switchably powered;
(f) the fan being removably mounted within the casing;
(g) the heating device being removably mounted within the casing;
(h) the heating device and the fan being positioned such that the fan is capable of blowing air toward the heating device;
(i) the discharge end having an opening;
(j) the heating device being capable of producing heat energy from an energy source chosen from the group including electrical, gas, solar, nuclear, and chemical;
(k) a means for directing the air through the opening and onto a wound;
(l) the air being capable of being increased in temperature thereby accelerating the healing of the wound;
(m) the air being capable of increased flow thereby diminishing moisture and sanitizing the wound;
(n) the casing having a length between 4 and 9 inches.
